(19) United States
(12) Patent Application Publication
(10) Pub. No.: US 2019/0117713 A1
(43) Pub. Date: Apr. 25, 2019

(54) ANTIBACTERIAL COMPOSITION

(71) Applicant: FermBiotics Holding ApS, Bække (DK)

(72) Inventor: Jens Høffner Legarth, Bække (DK)

(21) Appl. No.: 16/096,913

(22) PCT Filed: Apr. 28, 2017
(86) PCT No.: PCT/EP2017/060224

§ 371 Date: Oct. 26, 2018

(30) Foreign Application Priority Data
Apr. 28, 2016 (DK) PA 2016 00251

(51) Int. Cl. A61K 36/02; A61P 31/04; C12N 1/12; C12N 1/20

(52) U.S. Cl. CPC A61K 36/02; C12N 1/20; C12N 1/12; A61P 31/04

(57) ABSTRACT

The present invention relates to an antibacterial composition comprising a seaweed material having an average particle below 5 mm for treating, alleviating and/or preventing growth of gram-positive bacteria, wherein the gram-positive bacteria preferably is Clostridium bacteria or streptococcus bacteria.

### TECHNICAL FIELD OF THE INVENTION

The present invention relates to an antibacterial composition comprising seaweed. In particular, the present invention relates to an antibacterial composition comprising a seaweed material for treating, alleviating and/or preventing growth of gram-positive bacteria.

### BACKGROUND OF THE INVENTION

Six gram-positive genera are typically considered pathogenic in humans. These are Streptococcus, Staphylococcus, which are cocci (sphere-shaped) and the remaining organisms are bacilli (rod-shaped) and can be subdivided based on their ability to form spores, where the non-spore formers are Corynebacterium and Listeria (a coccobacillus), whereas Bacillus and Clostridium produce spores. The spore-forming bacteria can again be divided based on their respiration: Bacillus is a facultative anaerobe, while Clostridium is an obligate anaerobe.

One of the problems with these gram-positive bacteria is that they may have serious or pathogenic effects in humans and animals and is capable of causing serious and sometimes fatal infections in newborn infants.

Some of the implications and diseases in humans caused by gram-positive bacteria, in particular clostridium, may be:
Botulism; is a rare and potentially fatal illness caused by a toxin produced by the bacterium Clostridium botulinum present in air, food or in growing in wounds. The disease begins with weakness, trouble seeing, feeling tired, and trouble speaking. This may then be followed by weakness of the arms, chest muscles, and legs. The disease does not usually affect consciousness or cause a fever. The botulinum toxin is also known as Botox and is used in cosmetic surgery to paralyze facial muscles to reduce the signs of aging; it also has numerous other therapeutic uses.
Infection by Clostridium difficile can flourish when other gut flora bacteria are killed during antibiotic therapy, leading to superinfection and potentially fatal pseudomembranous colitis (a severe necrotizing disease of the large intestine).
Infection by Clostridium perfringens may cause a wide range of symptoms, from food poisoning to cellulitis, fasciitis, and gas gangrene.
Infection by Clostridium tetani may cause tetanus.
Infection by Clostridium sordellii can cause a fatal infection in exceptionally rare cases after medical abortions.

In general, the treatment of clostridial infection is high-dose penicillin G, to which the organism has remained susceptible.

Another gram-positive bacterium with pathogenic character and causing other implications and diseases in humans are streptococcus bacteria.

Streptococcus bacteria belongs to one of the most common agents resulting in infections in humans. Many different species of Streptococcus bacteria exist and they are divided into different sub-groups, where the most common groups are Group A, B, C and D.

In the medical setting, the most interesting groups are the alpha-hemolytic Streptococci pneumoniae and Streptococcus viridans group, and the beta-hemolytic streptococci of Lancefield groups A and B (also known as “GAS” and “BAS”).

The sub-group A (GAS) relates to the Streptococcus bacteria normally found on the skin and throat of humans and may result in various pathogenic infections.

These infections by GAS may be noninvasive or invasive. The noninvasive infections tend to be more common and less severe. The most common of these infections include streptococcal pharyngitis (strep throat) and impetigo. Scarlet fever is also a noninvasive infection, but has not been as common in recent years.

The invasive infections caused by GAS tend to be more severe and less common. This occurs when the bacterium is able to infect areas where it is not usually found, such as the blood and the organs. The diseases that may be caused include streptococcal toxic shock syndrome, necrotizing fasciitis, pneumonia, and bacteremia.

Globally, GAS has been estimated to cause more than 500,000 deaths every year, making it one of the world's leading pathogens.

Further complications that may be caused by GAS, may be acute rheumatic fever and acute glomerulonephritis. Rheumatic fever, is a disease that affects the joints, kidneys, and heart valves, and is a consequence of untreated GAS infection caused, and not by the bacterium itself. Rheumatic fever is caused by the antibodies created by the immune system to fight off the infection cross-reacting with other proteins in the body. This “cross-reaction” causes the body to essentially attack itself and leads to several damages.

However, there is an interest for the consumer, for the doctors, and for the society to limit or even omitting penicillin to avoid bacterial resistance; and other side effects associated with taken penicillin and in particular, when repeatedly taking penicillin.

Hence, an improved composition having antibacterial activity against gram-positive bacteria would be advantageous, and in particular a composition that does not have the same disadvantages and/or side effects as traditional compositions and medicines would be advantageous.

### SUMMARY OF THE INVENTION

Thus, an object of the present invention relates to a composition or an ingredient having antibacterial activity against gram-positive bacteria.

In particular, it is an object of the present invention to a composition or an ingredient having antibacterial activity against gram-positive bacteria and that solves the above mentioned problems of the prior art with bacterial resistance; and other side effects associated with traditionally used treatments and medication.

Thus, one aspect of the invention relates to an antibacterial composition comprising a seaweed materia for treating, alleviating and/or preventing growth of gram-positive bacteria.

Another aspect of the present invention relates to an antibacterial ingredient and/or an antibacterial composition comprising a seaweed material having an average particle size below 5 mm.

Yet another aspect of the present invention relates to a method of providing the antibacterial composition according to the present invention, and/or an antibacterial ingredient according to the present invention and/or the antibacterial composition according to the present invention, said method comprises the step of:
(i) Providing a seaweed material;
(ii) Reducing the size of the seaweed material;
(iii) Optionally, drying the seaweed material from step (ii); and
   providing the antibacterial composition and/or the antibacterial ingredient.

### DETAILED DESCRIPTION OF THE INVENTION

Accordingly, the inventor of the present invention surprisingly found that seaweed showed to poses an antibacterial activity against the growth and/or survival of gram-positive bacteria. Based on this finding an antibacterial ingredient and an antibacterial composition was developed which exhibited antibacterial activity against the growth and/or survival of gram-positive bacteria.

A preferred embodiment of the present invention relates to an antibacterial composition comprising a seaweed material for treating, alleviating and/or preventing growth of gram-positive bacteria. Preferably, the gram-positive bacteria are pathogenic gram-positive bacteria, even more preferably, the gram-positive bacteria or the pathogenic gram-positive bacteria are a cocci bacteria (sphere-shaped) or a bacilli bacteria (rod-shaped), even more preferably:
(i) the cocci bacteria are selected from the group consisting of Streptocuccus; and Staphylococcus, and/or
(ii) the bacilli bacteria are selected from the group consisting of Clostridium, Corynebacterium, Listeria, and bacillus

Preferably, the gram-positive bacteria are selected from the group consisting of Clostridium, Streptocuccus; Staphylococcus, Corynebacterium, Listeria, and bacillus, preferably, Clostridium and/or Streptocuccus.

In order to improve the antibacterial activity of the antibacterial ingredient and/or the antibacterial composition the seaweed material may comprise mechanically treated seaweed. In this way, the cell wall of the seaweed material may be broken, the cells are opened and intracellular liquid and intracellular components may be released and become more available for the consumer.

In an embodiment of the present invention the seaweed material may be subjected to a mechanical treatment. The mechanical treatment relates to any process suitable for reducing the size of the seaweed.

The mechanical treatment may preferably involve grinding, pulverization, chopping, lapping, cutting, or milling.

The mechanical treatment may be continued until the seaweed material reaches an average particle size below 5 mm, such as an average diameter below 4 mm, such as an average diameter below 3 mm, such as an average diameter below 2 mm, such as an average diameter below 1 mm, such as an average diameter below 500 μm, such as an average diameter below 250 μm, such as an average diameter below 100 μm, such as an average diameter below 50 μm, such as an average diameter in the range 25 μm to 5 mm, such as 0.1 mm to 4 mm, such as an average diameter in the range of 0.5 mm to 2.5 mm, such as an average diameter in the range 0.5 mm to 2 mm.

Hence, in an embodiment of the present invention the antibacterial ingredient and/or the antibacterial composition comprises a seaweed material having an average particle size below 5 mm, such as an average diameter below 4 mm, such as an average diameter below 3 mm, such as an average diameter below 2 mm, such as an average diameter below 1 mm, such as an average diameter below 500 μm, such as an average diameter below 250 μm, such as an average diameter below 100 μm, such as an average diameter below 50 μm, such as an average diameter in the range 25 μm to 5 mm, such as 0.1 mm to 4 mm, such as an average diameter in the range of 0.5 mm to 2.5 mm, such as an average diameter in the range 0.5 mm to 2 mm.

The inventors of the present invention surprisingly discovered that preparation of the antibacterial ingredient or the antibacterial composition may be kept simple and inexpensive without losing the antibacterial effect. In an embodiment of the present invention, it may be possible to provide an effective antibacterial ingredient and/or an effective antibacterial composition according to the present invention without using fermentation; chemical treatment and/or enzymatic treatment.

In an embodiment of the present invention the seaweed material may not been subjected to one or more of the treatments selected from fermentation by one or more lactic acid bacteria; to chemical treatment and/or enzymatic treatment.

In a further embodiment of the present invention the seaweed material may not been subjected to fermentation by any bacteria.

Without being bound by any theory the inventor of the present invention trusts that the antibacterial effect is obtained from a complex interaction of various components preset in the seaweed. Treatments like fermentation, chemical treatment, and/or enzymatic treatment may interact and influence on the structure and composition of these components in an unintended and undesirable manner.

In an embodiment of the present invention the seaweed material may be un-fractionated. In the present context the term “un-fractionated” relates to intact seaweed material which is used. The un-fractionated seaweed material also includes seaweed material obtained after removal of water, dead or unwanted material, and/or polluting material.

In another embodiment of the present invention the seaweed material may be fractionated into a fraction, an extract or an isolate having increased activity relative to the seaweed material as such.

In an embodiment of the present invention the seaweed material may relate to un-fractionated seaweed, a fraction obtained from seaweed, an isolate obtained from seaweed material or an extract obtained from seaweed. Preferably, seaweed material relates to un-fractionated seaweed.

The antibacterial effect may be achieved with the seaweed material as the only active antibacterial component. Therefore, the antibacterial ingredient and/or the antibacterial composition only comprise plant materials from below the water surface, e.g. a sub-sea plant material, such as seaweed and/or algae. Hence, the antibacterial ingredient and/or the antibacterial composition does not comprise a super-sea plant material.

In the present context, the term “seaweed” encompasses macroscopic, multicellular, benthic marine algae. The term includes members of the red, brown and green algae. Algae are a very large and diverse group of simple, typically autotrophic organisms, ranging from unicellular to multicellular forms, such as the giant kelps that grow to 65 meters in length. Seaweed materials according to the present invention may be photosynthetic and “simple” because they lack the many distinct cell and organ types found in land plants.

In an embodiment of the present invention the seaweed material may be an alga, preferably, the alga may be a red, brown and green alga, most preferably, the alga may be a brown alga. In a further embodiment of the present invention the brown alga may be selected from Laminariaceae; Laminariales or a combination hereof.

In yet an embodiment of the present invention the Laminariaceae may be selected from Laminaria digitata; Saccharina latissima; Laminaria hyperborean; Laminariagracilaria or a combination hereof.

In an even further embodiment of the present invention the Laminariales may be Alaria esculenta.

The antibacterial ingredient and/or the antibacterial composition may be supplemented with other ingredients, but preferably the seaweed material is the main component of the antibacterial ingredient and/or the antibacterial composition.

In an embodiment of the present invention the antibacterial ingredient and/or the antibacterial composition comprises at least 10% (w/w) of seaweed, such as at least 20%, e.g. at least 30%, such as at least 35%, e.g. at least 40%, such as at least 45%, e.g. at least 50%, such as at least 55%, e.g. at least 60%, such as at least 65%, e.g. at least 70%, such as at least 75%, e.g. at least 80%, such as at least 85%, e.g. at least 90%, such as at least 95%, e.g. about 100%.

The antibacterial ingredient and/or the antibacterial composition may be provided in any consumer preferred form. In an embodiment of the present invention the antibacterial ingredient and/or the antimicrobial composition may be in the form of a suspension, a slurry or a dry product

Due to the improved shelf life of the antibacterial ingredient and/or the antibacterial composition may be provided in the form of a dry powder. The form of a dry powder has also shown to be beneficial for maintaining the antibacterial activity of the antibacterial ingredient and/or the antibacterial composition.

In a preferred embodiment of the present invention an antibacterial ingredient may be provided. The antibacterial ingredient may be mixed with other ingredients (e.g. ingredients having antibacterial activity, antifungal activity, or the like) or it may be added to the antibacterial compound as the only active ingredient (e.g. the only ingredient showing antimicrobial activity.

In a preferred embodiment of the present invention the antibacterial ingredient comprising seaweed material having an average particle size below 5 mm.

Preferably, the seaweed material has not been subjected to one or more of the treatments selected from fermentation by one or more lactic acid bacteria; to chemical treatment and/or enzymatic treatment.

The antibacterial ingredient and/or the antibacterial composition according to the present invention may be prepared a method comprising the step of:
(i) Providing a seaweed material;
(ii) Reducing the size of the seaweed material;
(iii) Optionally, drying the seaweed material from step (ii); and
providing the antibacterial composition and/or the antibacterial ingredient.

In a preferred embodiment of the present invention the drying of the seaweed material is conducted as described in WO 2013/029632, which is hereby incorporated by reference.

In order to maintain the antibacterial activity of the antibacterial composition and/or the antibacterial ingredient, the seaweed, preferably during steps (i) to (iii); the antibacterial composition and/or the antibacterial ingredient is kept at a temperature during the method and/or during storage at 30° C. or below, such as 25° C. or below, e.g. 22° C. or below, such as 20° C. or below, e.g. 18° C. or below, such as 15° C. or below, e.g. 12° C. or below, such as 10° C. or below, e.g. 8° C. or below.

In a further embodiment of the present invention the seaweed, preferably during steps (i) to (iii); the antibacterial composition and/or the antibacterial ingredient may be subjected to a temperature above 65° C., such as above 75° C., e.g. above 80° C., such as above 85° C., e.g. above 90° C., for a period of less than 45 minutes, such as a period of less than 30 minutes, e.g. a period of less than 15 minutes, such as a period of less than 10 minutes a period of less than, e.g. a period of less than 5 minutes.

may not be subjected to a temperature above 90° C., such as above 85° C., e.g. above 80° C., such as above 75° C., e.g. above 70° C., such as above 65° C., e.g. above 60° C., such as above 55° C., e.g. above 50° C., for a period of 45 minutes or more, such as 30 minutes or more, e.g. 15 minutes or more, such as 10 minutes or more, e.g. 5 minutes or more.

Even it is preferred that the seaweed; the antibacterial composition and/or the antibacterial ingredient is not subjected to increased temperature for extended periods of time, the seaweed; the antibacterial composition and/or the antibacterial ingredient may be subjected to ultra-high temperature processing (UHT), ultra-heat treatment, and/or ultra-pasteurization, in order to kill microorganisms, like fungus, bacteria, yeast etc.

In an embodiment of the present invention the seaweed material may not be subjected to any fractionation steps, except removal of water, dead or unwanted material, or polluting materials.

In an embodiment of the present invention the seaweed material may be subjected to fractionation providing one or more isolates, fractions and/or extractions before providing the antibacterial composition and/or the antibacterial ingredient.

The seaweeds according to the method of the invention may also be stored for longer periods before handling. During such storage the seaweed material may be conserved, the method of conserving the seaweed material may however not involve fermentation of the seaweed; chemical treatment of the seaweed; or enzymatic treatment of the seaweed. One way to conserve the seaweed material could be to store it at cold temperatures reducing any spoiling activities of the seaweed.

Additionally, since seaweed farms of natural reasons often are positioned off-shore it would be advantageous also to be able to provide the antibacterial ingredient and/or the antibacterial composition off-shore, since it would otherwise be difficult to initiate the method before the harvested seaweed/algae starts to decompose due to contamination of undesired microorganisms.

Thus, in an embodiment the method for providing the antibacterial ingredient and/or the antibacterial composition may be carried out off-shore, such as on a ship, on a barge, or on a on an offshore platform.

In an embodiment of the present invention a super-sea plant material is added to the seaweed material.

The seaweed material and/or the super-sea plant material may be subjected to a fermentation process. Preferably, the fermentation process is performed using one or more lactic acid bacteria.

The fermentation process may be a fermentation of the seaweed material and the super-sea plant material individually, a co-fermentation process of the seaweed material and the super-sea plant material or a semi co-fermentation process of the seaweed material and the super-sea plant material.

In the present context, the term “co-fermentation process” relates to mixing the seaweed material and the super-sea plant material before any fermentation process has started. Whereas the term “semi co-fermentation process” relates to initial fermentation of one of the seaweed material or the super-sea plant material before the other material is added to the fermentation process. Preferably, the semi co-fermentation process starts fermenting the seaweed material before the super-sea plant material is added.

As mentioned above the harvested seaweed material may also be conserved off-shore and subsequently the fermentation takes place under controlled conditions in a factory facility.

In an embodiment of the present invention wherein the antibacterial composition comprises a fermented seaweed material, resulting in a fermented antibacterial composition and/or the antibacterial ingredient comprises a fermented seaweed material, resulting in a fermented antibacterial ingredient.

In a preferred embodiment of the present invention, the antibacterial ingredient and/or the antibacterial composition according to the present invention may comprise a fermented seaweed material for treating, alleviating and/or preventing growth of streptococcus bacteria.

A preferred aspect of the present invention relates to a fermented antibacterial ingredient and/or a fermented antibacterial composition comprising a fermented seaweed material for treating, alleviating and/or preventing growth of streptococcus bacteria.

Since the fermented antibacterial ingredient and/or the fermented antibacterial composition is suitable for treating, alleviating and/or preventing growth of streptococcus bacteria, one or more indications resulting from streptococcus induced infections may be treated by the fermented antibacterial ingredient and/or the fermented antibacterial composition according to the present invention.

In an embodiment of the present invention the streptococcus induced infections may include skin and soft tissue infections streptococcal pharyngitis (strep throat) and bullous impetigo. Scarlet fever; pneumonia; meningitis; bacteraemia (bacteria in the blood stream); sepsis; endocarditis (infection of the heart tissue); osteomyelitis; streptococcal toxic shock syndrome (toxic shock syndrome; TSST); necrotizing fasciitis; acute rheumatic fever; acute glomerulonephritis; or food poisoning.

The antibacterial ingredient and/or the antibacterial composition may comprise one or more super-sea plant materials or one or more fermented super-sea plant material. Preferably, the fermented antibacterial ingredient and/or the fermented antibacterial composition may comprise other ingredients, and comprises one or more super-sea plant materials or one or more fermented super-sea plant material, preferably another fermented super-sea plant material.

In an embodiment of the present invention the fermented antibacterial ingredient and/or the fermented antibacterial composition comprises at least 10% (w/w) of seaweed, such as at least 20%, e.g. at least 30%, such as at least 35%, e.g. at least 40%, such as at least 45%, e.g. at least 50%, such as at least 55%, e.g. at least 60%, such as at least 65%, e.g. at least 70%, such as at least 75%, e.g. at least 80%, such as at least 85%, e.g. at least 90%, such as at least 95%, e.g. about 100%.

In an embodiment of the present invention, the super-sea plant material may be selected from at least one proteinaceous plant material. The plant material, and the proteinaceous plant material, may be selected from at least one of Brassica spp.; sun flower; palm; soya, field beans, Lupins; or a combination hereof. Preferably, the plant material, and the proteinaceous plant material, may be selected from Brassica spp.

In an embodiment of the present invention, the Brassica spp. may preferably be selected from one or more of rape species; cruciferous vegetables; cabbage species; and/or mustard species. Preferably, the Brassica spp. may preferably be selected from one or more of rape species. Preferably, the rape species is a rapeseed product, such as rapeseed meal, or rapeseed cake, preferably rapeseed cake.

In another embodiment of the present invention, the Brassica spp. may be selected from one or more species such as Brassica napus; Brassica oleracea; Brassica campestris; and/or Brassica rapa.

The fermented antibacterial composition and/or the fermented antibacterial ingredeint according to the present invention may comprise one or more lactic acid bacterial strain(s).

In an embodiment of the present invention the fermented composition comprises one or more lactic acid bacterial strains, e.g. two or more lactic acid bacterial strains, such as three or more bacterial strains, e.g. four or more bacterial strains, such as 7 or more bacterial strains, e.g. 10 or more bacterial strains, such as 15 or more bacterial strains, e.g. 20 or more bacterial strains, such as 25 or more bacterial strains, e.g. 30 or more bacterial strains, such as 35 or more bacterial strains, e.g. 40 or more bacterial strains.

In a further embodiment of the present invention the one or more lactic acid bacterial strain(s) present in the fermented composition may be selected from the group consisting of Lactobacillus, Leuconostoc, Pediococcus, Lactococcus, Streptococcus, Aerococcus, Carnobacterium, Enterococcus, Oenococcus, Teragenococcus, Vagococcus, and Weisella. Preferably, the one or more lactic acid bacterial strain(s) present in the fermented composition are preferably lactic acid bacteria of the genus Enterococcus, Lactobacillus, Pediococcus, Lactococcus, or a combination thereof.

In an even further embodiment of the present invention the one or more lactic acid bacteria stain(s) may be selected from the group consisting of one or more Enterococcus spp., Lactobacillus spp., Lactococcus spp., Pediococcus spp., and a combination hereof. Preferably, the one or more lactic acid bacterial strain(s) is/are selected from the group consisting of one or more of Enterococcus faecium, Lactobacillus rhamnosus, Lactobacillus plantarum, Pediococcus acidililactili, Pediococcus pentosaceus, Lactococcus Lactis, Lactococcus Cremoris, Lactococcus Diacetylactis, Leuconostoc Cremoris and a combination hereof.

In yet a further embodiment of the present invention, the main lactic acid bacterial strain(s) present in the composition may be Pediococcus pentosaceus; Pendiococcus acidilactici; Lactobacillus plantarum; Lactobacillus rhamnosus; or Enterococcus faecium. Preferably, the main lactic acid bacteria present in the composition may be Lactobacillus plantarum.

In another embodiment of the present invention the one or more lactic acid bacteria stain(s) may be selected from the group consisting of one or more of Enterococcus faecium MCIMB 30122, Lactobacillus rhamnosus NCIMB 30121, Pediococcus pentosaceus HTS (LMG P-22549), Pendiococcus acidilactici NCIMB 30086 and/or Lactobacillus plantarum LSI (NCIMB 30083).

In order to provide the desired effects, the fermented composition should have a high content of viable lactic acid bacteria. In an embodiment of the present invention the fermented composition comprises one or more lactic acid bacterial strain(s) in a total amount in the range of 10⁵-10¹² CFU per gram, such as in the range of 10⁶-10¹² CFU per gram, e.g. in the range of 10⁷-10¹¹ CFU per gram, such as in the range of 10⁸-10¹¹ CFU per gram, e.g. in the range of 10⁹-10¹⁰ CFU per gram.

In an embodiment of the present invention the process of perform the fermentation process may be as described in PCT/EP2016/076952, which is hereby incorporated by reference.

It should be noted that embodiments and features described in the context of one of the aspects of the present invention also apply to the other aspects of the invention.

All patent and non-patent references cited in the present application, are hereby incorporated by reference in their entirety.

The invention will be further described in more detail in the following non-limiting examples.

### EXAMPLES

### Example 1

An antibacterial composition comprising a seaweed material, Laminaria digitate, comprising an average particle below 5 mm.

The antibacterial composition used in this experiment was subjected to grinding providing an average particle size of about 1 mm and suspended in water providing a concentration of 0.25 g/ml. After vigorously stirring the supernatant was used in the experiment. The seaweed material was not subjected to any treatments like fermentation by one or more lactic acid bacteria; to chemical treatment and/or enzymatic treatment.

Another plant material, comprising rape seed, was provided, grinded and suspended water providing a concentration of 0.25 g/ml. After vigorously stirring the supernatant was used in the experiment

The gram-positive bacteria, Clostridium bacteria, Clostridium perfringens (approximately 10⁶ CFU 7 ml), was provided in a growth medium and added to several wells of an ELISA plate, providing a McFarland turbidity of about 0.5. Different concentrations of the antibacterial compositions were quickly added to the various ELISA wells and incubated for about 24 hours at 37° C. Growth, or inhibition of growth of the Clostridium perfringens, was then observed.

From visual inspection, the results showed that the antibacterial composition comprising seaweed material, Laminaria digitate, having an average particle below 5 mm, was suitable for treating, alleviating and/or preventing growth of gram-positive bacteria, illustrated by Clostridium perfringens, even at low concentrations, whereas growth of Clostridium perfringens, was continued in the experiments comprising rape seed as well as the experiments comprising the control samples (neither comprising seaweed nor rape seed material).

### Example 2

This example illustrates a method according to the present invention for providing a fermented antibacterial composition comprising a combination of 20% seaweed (Laminaria saccharine) and 80% rapeseed meal.

Seaweed (Laminaria saccharina), was pre-treated to an average diameter of about 1 mm, and mixed with a combination of the lactic acid-producing bacteria Pediococcus pentosaceus, Pediococcus acidilactici and Lactobacillus plantarum in a fermentation tank and the moisture content was adjusted to about 40% humidity. The air intake to the fermentation tank was taken directly from the air in the room wherein the fermentation tank was situated. The fermentation was performed at a temperature of about 30° C.

After 3 days of fermentation of the seaweed, rapeseed meal, which was pre-treated to about 2 mm, was added to the fermented composition comprising seaweed, in a ratio of rapeseed meal and seaweed is 80:20 (on a weight basis), together with a combination of the lactic acid-producing bacteria Pediococcus pentosaceus, Pediococcus acidilactici and Lactobacillus plantarum in a fermentation tank and the fermentation was continued for 3 days at 25° C. The resulting fermentation broth had a bacterial count of 3.0×10⁶ CFU/gram, a pH of 4.2 and a lactic acid concentration of about 140 mM.

The resulting fermented antibacterial composition comprising the combination of seaweed, rapeseed meal and lactic acid bacteria, was subjected to spin flash drying in order to protect the lactic acid bacteria and keep a high number of viable cells.

### Example 3

A patient, 55 years old male, suffering from a streptococcus infection diagnosed by the medical doctor and by analysing a blood sample from the patient. The infection was resulting in serious swelling of the lower legs, blushing, strong pain in the legs and difficulties in walking and wearing socks and shoes.

The patient was giving a penicillin (800 mg phenoxymethylpenicillinsodium), 1 pill 3 times a day for 10 days. After a first treatment, no effect of the penicillin treatment was observed.

The treatment with penicillin was repeated 4 times more and no significant improvement of change in the streptococcus infection was observed.

The patient started taken a single daily dose of approx. 15 gram (a tea spoon) of the dry fermented antimicrobial composition obtained in Example 2. After taken a single daily dose of approx. 15 gram for 7 days the swelling was significantly reduced and the patient experienced significantly less pain. After 10 days of taken a single daily dose of approx. 15 gram the swelling was gone, there were no more pain and the blood analysis showed that the streptococcus infection was gone.

The only signs remaining from the infection was the formation of scales on the skin of the leg due to the serious swelling.

The intake of the dry fermented antimicrobial composition obtained in Example 2 was stopped and after 6 months no relapse was observed.

### REFERENCES

WO 2013/029632

1. An antibacterial composition comprising a seaweed material for treating, alleviating and/or preventing growth of gram-positive bacteria.

2. The antibacterial composition according to claim 1, wherein the gram-positive bacteria is Clostridium bacteria or streptococcus bacteria.

3. The antibacterial composition according to claim 1, wherein the seaweed material has an average particle below 5 mm.

4. The antibacterial composition according to claim 1, wherein the antibacterial composition comprises at least 10% (w/w) of seaweed.

5. (canceled)

6. The antibacterial composition according to claim 1, wherein the seaweed material has not been subjected to one or more of the treatments selected from fermentation by one or more lactic acid bacteria; to chemical treatment and/or enzymatic treatment.

7. The antibacterial composition according to claim 1, wherein the antibacterial composition comprises a fermented seaweed material, resulting in a fermented antibacterial composition.

8. The antibacterial composition according to claim 7, wherein the fermented antibacterial composition comprises one or more lactic acid bacterial strain(s).

9. (canceled)

10. The antibacterial composition according to claim 1, wherein the antibacterial composition comprises a super-sea plant material.

11. (canceled)

12. The antibacterial composition according to claim 10, wherein the super-sea plant material is a fermented super-sea plant material.

13. An antibacterial ingredient comprising a seaweed material having an average particle size below 5 mm.

14. The antibacterial ingredient according to claim 13, wherein the seaweed material is mechanically treated seaweed, wherein the mechanical treatment relates to any process reducing the size of the seaweed.

15. The antibacterial ingredient according to claim 13, wherein the antibacterial ingredient comprises at least 10% (w/w) of seaweed.

16. The antibacterial ingredient according to claim 13, wherein the seaweed material has not been subjected to one or more of the treatments selected from fermentation by one or more lactic acid bacteria; to chemical treatment and/or enzymatic treatment.

17. The antibacterial ingredient according to claim 13, wherein the antibacterial ingredient comprises a fermented seaweed material, resulting in a fermented antibacterial ingredient.

18. The antibacterial ingredient according to claim 17, wherein the fermented antibacterial ingredient comprises one or more lactic acid bacterial strain(s).

19. (canceled)

20. The antibacterial ingredient according to claim 13, wherein the antibacterial ingredient comprises a super-sea plant material.

21. (canceled)

22. (canceled)

23. The method of providing the antibacterial composition according to claim 1, said method comprises the step of:
(i) Providing a seaweed material;
(ii) Reducing the size of the seaweed material;
(iii) Optionally, drying the seaweed material from step (ii); and
providing the antibacterial composition.

24. (canceled)

25. The method according to claim 23, wherein the seaweed material and/or the antibacterial composition is subjected to ultra-high temperature processing (UHT), ultra-heat treatment, and/or ultra-pasteurization.

26. The method according to claim 23, wherein the seaweed material is not subjected to any fractionation steps except removal of water, dead or unwanted material, or polluting material.

27. (canceled)

28. The method according to claim 23, wherein a super-sea plant material is added to the seaweed material.

29.-31. (canceled)
